(19) United States
(12) Patent Application Publication
(10) Pub. No.: US 2026/0275070 A1
(43) Pub. Date: Sep. 17, 2026

(54) METHOD FOR DETERMINING INPUT AMOUNT OF SODIUM HYDROXIDE

(71) Applicant: HONDA MOTOR CO., LTD., Tokyo (JP)

(72) Inventors: Keishi ASADA, Wako-shi, Saitama (JP); Ryo NAKAI, Wako-shi, Saitama (JP)

(21) Appl. No.: 19/552,610

(22) Filed: Feb. 27, 2026

(30) Foreign Application Priority Data
Mar. 11, 2025 (JP) 2025-038670

(51) Int. Cl. C08J 11/16; G01N 33/44

(52) U.S. Cl. CPC C08J 11/16; G01N 33/442

(57) ABSTRACT

A method for determining an input amount of sodium hydroxide is provided, which can determine an appropriate input amount of sodium hydroxide when depolymerizing plastic composed of multiple types of resins. The method contributes to a significant reduction in generating waste. The appropriate input amount is determined by using a map including a graph having a plurality of lines. In the map, a horizontal axis represents a remaining amount of sodium hydroxide, and a vertical axis represents a deficient amount of sodium hydroxide. The method includes steps of calculating an amount of ethylene glycol or an amount of butanediol and a remaining amount of sodium hydroxide after a predetermined time has elapsed from when a predetermined amount of sodium hydroxide is added to a predetermined amount of the plastic, and applying these calculated amounts to the map.

### CROSS-REFERENCE TO RELATED APPLICATION

This disclosure claims the priority benefit of Japanese Patent Application No. 2025-038670 filed on March 11, 2025, and the entirety of the above-mentioned patent application is hereby incorporated by reference herein and made a part of this specification.

### TECHNICAL FIELD

The present disclosure relates to a method for determining an input amount of sodium hydroxide.

### BACKGROUND ART

Recently, efforts have been made to significantly reduce generation of waste by preventing the generation of waste, reducing an amount of the waste, and recycling and reusing the waste. Directed to realization of these efforts, research and development on recycling of plastics have been conducted. For example, chemical recycling is performed in which a new plastic product is produced by chemically decomposing plastic waste and chemically synthesizing the new plastic product by using the decomposed waste. For decomposition of plastics, used is a method for depolymerizing the plastics by sodium hydroxide (for example, refer to Japanese Unexamined Patent Application Publication No.2023-163661).

### SUMMARY OF THE DISCLOSURE

Meanwhile, the technique relating to recycling of plastics has the following disadvantage. For example, in mixed plastic which is plastic composed of multiple types of resins, a composition ratio of the resins is different in each lot. Therefore, when the mixed plastic is depolymerized with sodium hydroxide, an amount of sodium hydroxide required varies. Therefore, there is a need for a method for determining an appropriate input amount of sodium hydroxide. Thus, it is an object of the present disclosure to develop a method for determining an appropriate input amount of sodium hydroxide when the mixed plastic is depolymerized.

In order to solve the above described disadvantage, the present application is directed to a method for determining an appropriate amount of sodium hydroxide when depolymerizing plastic composed of multiple types of resins. This method contributes to a significant reduction in generation of waste.

A method for determining an input amount of sodium hydroxide according to the present disclosure for solving the above described disadvantage is a method for determining an input amount of sodium hydroxide required for depolymerizing plastic composed of multiple types of resins by using a map having a graph of a plurality of lines. Herein, a horizontal axis representing a remaining amount of sodium hydroxide and a vertical axis representing a deficient amount of sodium hydroxide in the map. Further, the lines are shifted depending on an amount of ethylene glycol or an amount of butanediol in the graph. In the method, slurry is produced by adding a predetermined amount of sodium hydroxide to a predetermined amount of the plastic. The method includes a step of calculating an amount of ethylene glycol or an amount of butanediol in filtrate of the slurry after a predetermined time has elapsed, a step of calculating a remaining amount of sodium hydroxide in the filtrate of the slurry, and a step of calculating a deficient amount of sodium hydroxide by applying a value of the calculated amount of ethylene glycol or a value of the calculated amount of butanediol and a value of the calculated remaining amount of sodium hydroxide to the map.

According to a method for determining an input amount of sodium hydroxide of the present disclosure, it is possible to determine an appropriate input amount of sodium hydroxide when depolymerizing plastic composed of multiple types of resins.

### BRIEF DESCRIPTION OF THE DRAWINGS

FIG. 1 is a diagram showing a map used in a method for determining an input amount of sodium hydroxide in an embodiment.

FIG. 2 is a flowchart showing a method for determining an input amount of sodium hydroxide in an embodiment.

### DETAILED DESCRIPTION OF EMBODIMENTS

Hereinafter, embodiments of the present disclosure will be described in detail, but the embodiments of the present disclosure are not limited to the embodiments described below.

A method for determining an input amount of sodium hydroxide is a method for determining an input amount of sodium hydroxide required for depolymerizing (or decomposing) plastic composed of multiple types of resins (hereinafter, appropriately referred to as mixed plastic) by using a map having a graph of a plurality of lines. Here, the lines in the graph are, for example, straight lines, but may include a curved line or a bending point as necessary.

In the present embodiment, the mixed plastic is used which contains not only polyethylene terephthalate (PET) or polybutylene terephthalate (PBT), but also polyurethane (PU).

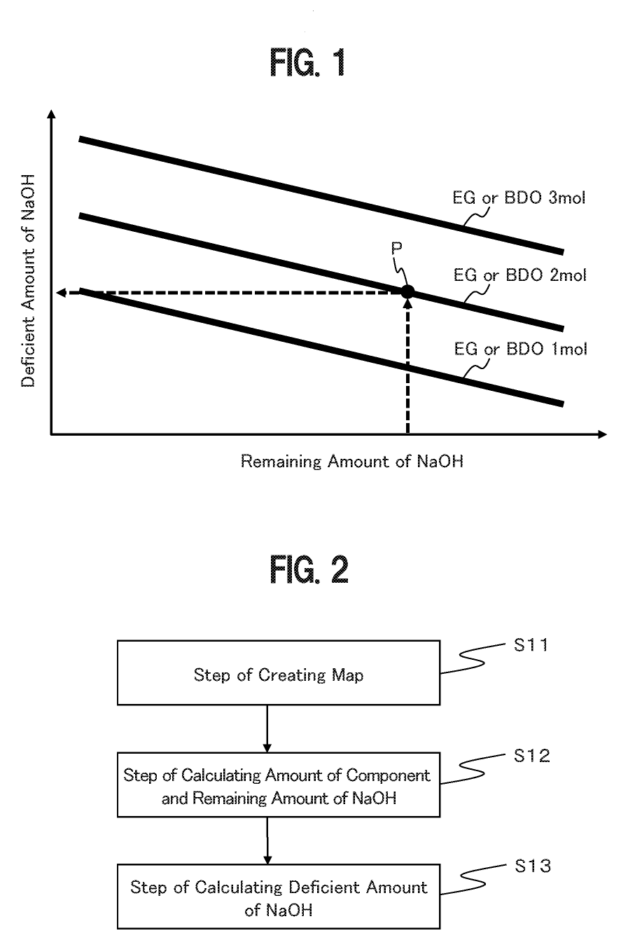

As shown in FIG. 1, the horizontal axis represents a remaining amount of sodium hydroxide (i.e., remaining amount of NaOH), and the vertical axis represents a deficient amount of sodium hydroxide (i.e., deficient amount of NaOH) in the map. The map here has a graph of three straight lines. The three straight lines in the graph are shifted depending on an amount of ethylene glycol (EG) or an amount of butanediol (BDO). Specifically, in the order of 1mol, 2mol, and 3mol, the three straight lines are formed from a lower side of the map with a predetermined inclination.

Here, the remaining amount of sodium hydroxide is an amount of sodium hydroxide remaining without being consumed in the depolymerization of the mixed plastic. Further, the deficient amount of sodium hydroxide is an amount of sodium hydroxide which is deficient and required in addition to an input amount of sodium hydroxide at the initial stage.

When the mixed plastic contains polyethylene terephthalate, used is a graph of straight lines based on an amount of ethylene glycol generated by depolymerizing polyethylene terephthalate. If the mixed plastic contains polybutylene terephthalate, used is a graph of straight lines based on an amount of butanediol generated by depolymerizing polybutylene terephthalate.

According to the method for determining an input amount of sodium hydroxide, an input amount of sodium hydroxide required for depolymerizing the mixed plastic is determined by applying values obtained by experiments as described later, to the map prepared in advance.

Hereinafter, an example of the present embodiment will be described more specifically.

As shown in FIG. 2, the present embodiment includes a step S11 of creating a map, a step S12 of calculating an amount of a component and a remaining amount of NaOH, and a step S13 of calculating a deficient amount of NaOH.

### Step of Creating Map

The step S11 of creating a map is, for example, a step of creating a map shown in FIG. 1 described above.

Hereinafter, a method for creating the map will be described. Here, a case where the three straight lines in the graph represent an amount of ethylene glycol (EG) will be described.

First, samples of the mixed plastic are prepared in which contents of polyethylene terephthalate (PET) and polyurethane (PU) are known. As the samples, prepared are a plurality of samples in which contents of polyethylene terephthalate are same but contents of polyurethane are different. An amount of sodium hydroxide (i.e., total amount of NaOH) required for depolymerizing polyethylene terephthalate and polyurethane in each sample is calculated in advance. Further, the content of the polyethylene terephthalate is adjusted so that an amount of ethylene glycol produced by decomposing polyethylene terephthalate becomes 1mol. The amount of ethylene glycol can be calculated by gas chromatography-mass spectrometry described later. Next, the same amount of sodium hydroxide (i.e., input amount of NaOH) which is a predetermined amount, is added into each sample. A remaining amount of sodium hydroxide in filtrate of slurry, for example, after 30 minutes have elapsed is calculated. The remaining amount of sodium hydroxide can be calculated by neutralization titration described later.

In each sample, a value obtained by subtracting the input amount of NaOH from the total amount of NaOH, that is, a value of the deficient amount of sodium hydroxide is plotted on the vertical axis, and the remaining amount of sodium hydroxide is plotted on the horizontal axis. A first straight line is drawn based on each plot.

By increasing the content of the polyethylene terephthalate and making the others the same as in the case of drawing the first straight line, a second straight line in which the amount of ethylene glycol generated by decomposing polyethylene terephthalate is set to 2mol and a third straight line in which the amount of ethylene glycol generated by decomposing polyethylene terephthalate is set to 3mol are drawn. The straight line of 2mol and the straight line of 3mol may be drawn based on the straight line of 1mol or may be drawn based on data in actual experiments.

Here, a case has been described where the mixed plastic contains polyethylene terephthalate and polyurethane and the graph of the three straight lines shows the amount of ethylene glycol. However, a map can be created by the same method even in a case where the mixed plastic contains polybutylene terephthalate (PBT) and polyurethane and the graph of the three straight lines represents the amount of butanediol (BDO).

Note, the created map can be applied to other lots of the mixed plastic for which deficient amounts of sodium hydroxide are calculated depending on conditions. Thus, once the map is created, the step S11 of creating a map can be appropriately omitted from the next time.

### Step of Calculating Amount of Component and Remaining Amount of NaOH

The step of S12 includes a step of calculating an amount of ethylene glycol or butanediol in filtrate of slurry after a predetermined time has elapsed (i.e., step of calculating an amount of a component) after adding a predetermined amount of sodium hydroxide to a predetermined amount of plastic to produce the slurry, and a step of calculating an remaining amount of sodium hydroxide in the filtrate of the slurry (i.e., step of calculating a remaining amount of NaOH). Here, the amount of the component herein refers to an amount of ethylene glycol generated by depolymerizing polyethylene terephthalate or an amount of butanediol generated by depolymerizing polybutylene terephthalate.

Hereinafter, an example of an experiment where values to be applied to these maps are calculated will be described.

For example, first, 40g of mixed plastic to be decomposed is prepared, and 0.05mol of sodium hydroxide is added as an initial input amount to the mixed plastic to produce slurry. After 30 minutes have elapsed after the slurry was produced, an amount of ethylene glycol or an amount of butanediol in filtrate of the slurry is calculated. Further, after 30 minutes have elapsed after the slurry was produced, a remaining amount of sodium hydroxide in the filtrate of the slurry is calculated.

When a graph of the map shows an amount of ethylene glycol, the amount of ethylene glycol in the filtrate of the slurry is calculated. Further, when a graph of the map shows an amount of butanediol, the amount of butanediol in the filtrate of the slurry is calculated.

Preferably, the amount of ethylene glycol or the amount of butanediol in the filtrate is determined by gas chromatography-mass spectrometry. The gas chromatography-mass spectrometry enables the amount of ethylene glycol or the amount of butanediol in the filtrate to be easily calculated.

In the gas chromatography-mass spectrometry, first, filtrate is neutralized with 1M sulfate while checking a pH by a pH test paper. Next, the neutralized filtrate is filtered through a syringe filter, and the resulting filtrate is analyzed by a gas chromatography-mass spectrometer so as to calculate the amount of ethylene glycol or the amount of butanediol.

Preferably, a remaining amount of sodium hydroxide in the filtrate is calculated by neutralization titration. That is, the remaining amount of sodium hydroxide in the filtrate can be easily calculated by using neutralization titration.

In the neutralization titration, phenolphthalein is first added to the filtrate. Next, neutralization titration with acetic acid is performed, and a point at which the solution becomes colorless is taken as an end point. Thereby, the remaining amount of sodium hydroxide is calculated.

Preferably, the filtrate is divided into first filtrate to be used for gas chromatography-mass spectrometry and second filtrate to be used for neutralization titration. Then, the first and second filtrate are diluted with a solvent. Diluting the filtrate with a solvent facilitates gas chromatography-mass spectrometry and neutralization titration. Further, the procedure improves accuracy thereof.

Further, in the neutralization titration, after filtration of the slurry, dilution of the filtrate with a solvent enables disodium terephthalate which is a product of reacted sodium hydroxide to be captured on a residue side of the filtration. Note, if the slurry is diluted with a solvent before the filtration, sodium hydroxide thus having reacted and been converted to disodium terephthalate enters the filtrate, making it difficult to quantify an amount of unreacted sodium hydroxide.

The solvent includes, for example, methanol, ethanol, and acetone in gas chromatography-mass spectrometry. Further, in the neutralization titration, the solvent includes, for example, water.

In the method for determining the amount of sodium hydroxide, the neutralization titration is preferably started after starting gas chromatography-mass spectrometry. Since the gas chromatography-mass spectrometry requires time, a total experiment time can be shortened by performing the gas chromatography-mass spectrometry first. This procedure can shorten the time for calculating a deficient amount of sodium hydroxide.

### Step of Calculating Deficient Amount of NaOH

A step S13 where a deficient amount of NaOH is calculated is a step of calculating the deficient amount of sodium hydroxide by applying the following values to a map. Herein, the values are a value of an amount of ethylene glycol or an amount of butanediol both calculated in the step S12 where the amount of the component and the remaining amount of NaOH are calculated, and a value of the remaining amount of sodium hydroxide thus calculated in the step S12 where the amount of the component and the remaining amount of NaOH are calculated.

Specifically, as shown in FIG. 1, values of the amount of ethylene glycol or the amount of butanediol calculated in the step S12, where the amount of component and the remaining amount of NaOH are calculated, are applied based on the three straight lines of the map, and values of the remaining amount of sodium hydroxide calculated in the step S12, where the amount of component and the remaining amount of NaOH are calculated, are applied to the horizontal axis of the map and plotted. Then, a value of the deficient amount of sodium hydroxide on the vertical axis of the map at the plotted position is confirmed. The value of the deficient amount of sodium hydroxide on the vertical axis of this map is a deficient amount of sodium hydroxide in the mixed plastic of a predetermined lot. In FIG. 1, as an example, a point P is plotted for a case where the calculated remaining amount of sodium hydroxide is a predetermined amount and the calculated amount of ethylene glycol or butanediol is 2mol. The value on the vertical axis at the plotted point P is a deficient amount of sodium hydroxide.

Calculating the deficient amount of sodium hydroxide enables an appropriate amount of sodium hydroxide to be used for the mixed plastic in a predetermined lot to be determined. That is, a sum of an initial input amount of sodium hydroxide and a deficient amount of sodium hydroxide is an appropriate amount of sodium hydroxide to be used for the mixed plastic in a predetermined lot.

As described above, according to the method for determining the amount of sodium hydroxide in the present embodiment, an appropriate input amount of sodium hydroxide when depolymerizing the mixed plastic can be simply and quickly determined.

Although the embodiments for carrying out the invention have been described above more specifically, the gist of the present disclosure is not limited to these descriptions, and should be broadly interpreted based on the description of the claims. Further, various changes, modifications, and the like based on the description are also included in the gist of the present invention.

For example, in the creation of the map, the content of polyethylene terephthalate was adjusted so that the amount of ethylene glycol generated by decomposing polyethylene terephthalate became 1mol, and the first straight line in which the amount of ethylene glycol became 1mol was drawn. However, the first straight line may be drawn based on the obtained amount of ethylene glycol without adjusting the content of polyethylene terephthalate so that the amount of ethylene glycol becomes 1mol.

### REFERENCE SIGNS LIST

Point P

What is claimed is:

1. A method for determining an input amount of sodium hydroxide required for depolymerizing plastic composed of multiple types of resins by using a map having a graph of a plurality of lines, wherein
a horizontal axis represents a remaining amount of sodium hydroxide and a vertical axis represents a deficient amount of sodium hydroxide in the map, and
the lines are shifted by an amount of ethylene glycol or an amount of butanediol in the graph,
the method comprising:
a step of calculating an amount of ethylene glycol or an amount of butanediol in filtrate of slurry after a predetermined time has elapsed, wherein the slurry is produced by adding a predetermined amount of sodium hydroxide to a predetermined amount of the plastic,
a step of calculating a remaining amount of sodium hydroxide in filtrate of the slurry, and
a step of calculating a deficient amount of sodium hydroxide by applying a value of the calculated amount of ethylene glycol or the calculated amount of butanediol and a value of the calculated remaining amount of sodium hydroxide, to the map.

2. The method for determining the input amount of sodium hydroxide according to claim 1, wherein the amount of ethylene glycol or the amount of butanediol in the filtrate is calculated by gas chromatography-mass spectrometry.

3. The method for determining the input amount of sodium hydroxide according to claim 1, wherein the remaining amount of sodium hydroxide in the filtrate is calculated by neutralization titration.

4. The method for determining the input amount of sodium hydroxide according to claim 2, wherein the remaining amount of sodium hydroxide in the filtrate is calculated by neutralization titration.

5. The method for determining the input amount of sodium hydroxide according to claim 1, wherein
the amount of ethylene glycol or the amount of butanediol in the filtrate is calculated by gas chromatography-mass spectrometry, and the remaining amount of sodium hydroxide in the filtrate is calculated by neutralization titration, and
the filtrate is diluted with a solvent after the filtrate is divided to first filtrate to be used for the gas chromatography-mass spectrometry and second filtrate to be used for the neutralization titration.

6. The method for determining the input amount of sodium hydroxide according to claim 1, wherein
the amount of ethylene glycol or the amount of butanediol in the filtrate is calculated by gas chromatography-mass spectrometry, and the remaining amount of sodium hydroxide in the filtrate is calculated by neutralization titration, and
the neutralization titration is started after starting the gas chromatography-mass spectrometry.
